(19) United States
(12) Patent Application Publication
(10) Pub. No.: US 2003/0163135 A1
(43) Pub. Date: Aug. 28, 2003

(54) Orthopeadic reamer with see-through viewing windows

(76) Inventor: Ray W. Hathaway, Pierceton, IN (US)

Correspondence Address: Todd T. Taylor, TAYLOR & AUST, P.C., 142 S. Main St., P.O. Box 560, Avilla, IN, 46710, US

(21) Appl. No.: 10/080,490

(22) Filed: Feb. 22, 2002

(51) Int. Cl. A61B 17/16

(52) U.S. Cl. 606/80

(57) ABSTRACT

An orthopeadic reamer for cutting bone includes a shaft and a head coupled with the shaft. The head includes a distal face with a plurality of cutting teeth and at least one viewing window. Each viewing window extends through the head.

### BACKGROUND OF THE INVENTION

1. Field of the Invention

The present invention relates to orthopeadic reamers, and, more particularly, to orthopeadic reamers having a distal cutting face.

2. Description of the Related Art

An orthopeadic reamer is used to cut a bone and thereby form the bone with a predetermined shape for receiving an orthopeadic implant. For example, an intramedullary reamer may be placed into the intramedullary canal of the bone and used to ream the interior of the bone to receive the stem of an orthopeadic implant. Such a reamer includes a radial, peripheral surface which generally includes a plurality of radially extending teeth for cutting the bone in a radial direction as the reamer proceeds in an axial direction into the bone. The size of the opening formed in the bone is determined by the outside diameter of the reamer.

An orthopeadic reamer may also include a cutting head with a distal face which has a plurality of cutting teeth formed therein. The distal face has a shape which corresponds to the shape of an orthopeadic implant to be received therein, and includes a plurality of generally axially extending teeth. The reamer is placed against the bone surface to be cut, such as an acetabulum or glenoid, and is plunge cut into the bone. Such reamers are effective for removing a portion of the bone so that the bone is shaped to receive the implant.

A problem with a reamer having generally axially facing cutting teeth is that the surgeon cannot easily see the bone surface being cut. Thus, it is usually necessary to stop rotation of the reamer and remove the reamer from the cutting site prior to viewing the bone. This process may be relatively tedious and has a possibility of further damaging soft tissue adjacent the cutting site.

What is needed in the art is an orthopeadic reamer which may be used for axial plunge cuts, and still allows a surgeon to view the bone without removing the reamer from the cutting site.

### SUMMARY OF THE INVENTION

The present invention provides an orthopeadic reamer including a cutting head with a plurality of viewing windows which extend therethrough to allow a surgeon to view the bone during an operation procedure.

The invention comprises, in one form thereof, an orthopeadic reamer for cutting bone, including a shaft and a head coupled with the shaft. The head includes a distal face with a plurality of cutting teeth and at least one viewing window. Each viewing window extends through the head.

The invention comprises, in another form thereof, a method of cutting bone using an orthopeadic reamer, including the steps of: providing an orthopaedic reamer, including a shaft and a head coupled with the shaft, the head including a distal face with a plurality of cutting teeth and at least one viewing window, each viewing window extending through the head; placing the orthopaedic reamer against the bone; rotating the orthopaedic reamer to thereby cut the bone using the plurality of teeth; stopping rotation of the orthopaedic reamer; and viewing the bone through at least one viewing window.

An advantage of the present invention is that a surgeon may view the bone being cut during a surgical procedure.

Another advantage is that the particular cross-sectional configuration of the viewing windows may vary from one application to another, and still provide adequate viewing of the bone being cut.

Yet another advantage is that the number as well as the cross-sectional shape of the viewing windows may vary from one application to another, and still provide adequate viewing of the bone being cut.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a side view of an embodiment of an orthopeadic reamer of the present invention;

FIG. 2 is an end view of the orthopeadic reamer of FIG. 1, as viewed from the cutting side of the head; and

FIG. 3 is a sectional view taken along line 3-3 in FIG. 1.

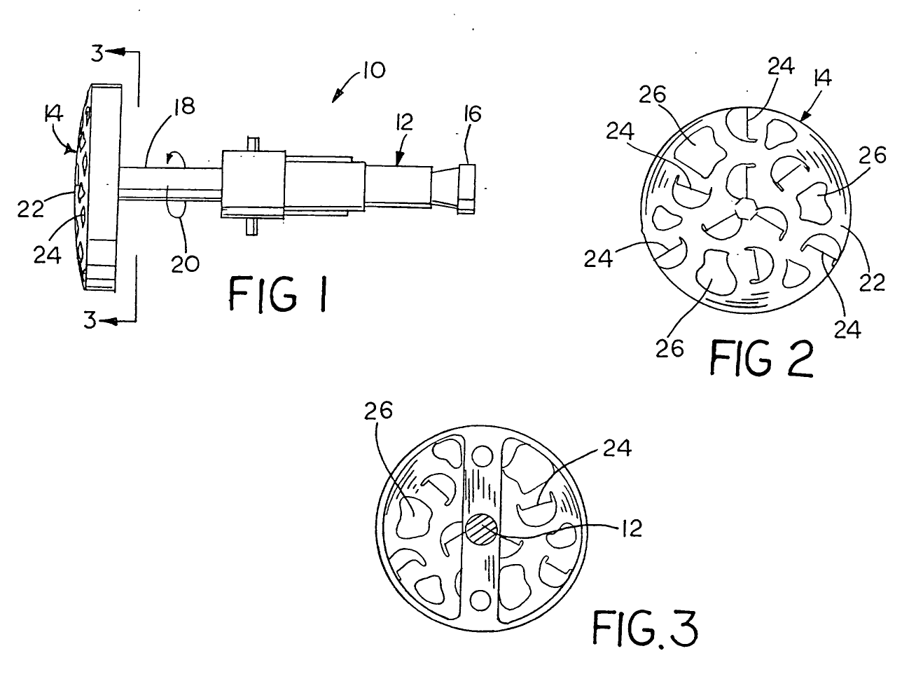

Corresponding reference characters indicate corresponding parts throughout the several views. The exemplification set out herein illustrates one preferred embodiment of the invention, in one form, and such exemplification is not to be construed as limiting the scope of the invention in any manner.

### DETAILED DESCRIPTION OF THE INVENTION

Referring now to the drawings, there is shown an embodiment of an orthopeadic reamer 10 of the present invention which is used for cutting bone. For example, orthopeadic reamer 10 may be used to cut bone at a shoulder, knee or hip joint, depending upon the particular configuration. Orthopeadic reamer 10 generally includes a shaft 12 and a head 14.

Shaft 12 includes a driven end 16 and a distal end 18. Driven end 16 is removably coupled with a source of rotational power for driving shaft 12 in a rotating manner, as indicated by arrow 20. Distal end 18 is coupled with head 14 in any suitable manner, such as welding, threaded engagement, etc.

Head 14 includes a distal face 22 which is placed against a bone to be cut, and includes a predetermined shape which is dependent upon a particular application. In the embodiment shown, distal face 22 has a generally hemispherical shape with a predetermined radius of curvature of approximately three inches for cutting a glenoid associated with a shoulder.

Head 14 also includes a plurality of cutting teeth 24 which are formed in distal face 22 by a stamping operation. Each cutting tooth 24 has a partial hemispherical shape as a result of the stamping operation. In particular, each cutting tooth 24 generally forms half of a hemisphere and defines a generally linear cutting edge for cutting bone.

Head 14 also includes one or more viewing windows 26, and preferably includes a plurality of viewing windows 26 as shown in the drawings. Each viewing window 26 extends through head 14 such that a user may view the bone through head 14, when viewed from the back side of head 14 during use.

Each viewing window 26 has a predetermined cross-sectional configuration which may be the same as or different from the cross sectional configuration of one or more other viewing windows 26. The cross-sectional configuration of each viewing window 26 is preferably constructed dependent upon a location of adjacent cutting teeth 24 so as not to interfere with the operation or structural integrity of head 14. That is, certain axial loading forces will be applied by a surgeon, as well as rotational forces will be applied against cutting teeth 24 as head 14 rotates during operation. Viewing windows 26 are preferably constructed so as to provide maximum viewing of the bone when head 14 is at a stand still position, while at the same time not compromising the structural integrity of head 14. In the embodiment shown, each viewing window 26 has a continuous compound curvature which is predefined, dependent upon the size, shape and location of adjacent cutting teeth 24. Thus, the size, shape and cross-sectional configuration of viewing windows 26 likewise may change from one application to another.

During an orthopeadic operation, head 14 of orthopeadic reamer 10 is placed against a bone (not shown) to be cut. Orthopeadic reamer 10 is then driven in a rotational direction 20 using a rotating driver (not shown). As orthopeadic reamer 10 rotates, cutting teeth 24 cut the bone using the plurality of cutting teeth 24. Rotation of head 14 is intermittently stopped so that the surgeon may view the bone through one or more viewing windows 26 to ascertain the extent to which the bone is cut. When the bone is properly prepared for an implant, orthopeadic reamer 10 is removed.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthopaedic reamer for cutting bone, comprising:
a shaft; and
a head coupled with said shaft, said head including a distal face with a plurality of cutting teeth and at least one viewing window, each said viewing window extending through said head.

2. The orthopaedic reamer of claim 1, said at least one viewing window comprising a plurality of viewing windows.

3. The orthopaedic reamer of claim 2, each said viewing window having a predefined cross-sectional configuration, at least two of said cross-sectional configurations being different.

4. The orthopaedic reamer of claim 1, said at least one viewing window having a cross-sectional configuration with a continuous compound curvature.

5. The orthopaedic reamer of claim 4, said cross-sectional configuration being dependent upon a location of said viewing window relative to adjacent said cutting teeth.

6. The orthopaedic reamer of claim 1, said at least one viewing window having a cross-sectional configuration which is dependent upon location of said viewing window relative to adjacent said cutting teeth.

7. The orthopaedic reamer of claim 1, said cutting teeth being generally partial hemispherical shaped.

8. The orthopaedic reamer of claim 1, said head having an overall generally hemispherical shape.

9. A method of cutting bone using an orthopaedic reamer, comprising the steps of:
providing an orthopaedic reamer, including:
a shaft; and
a head coupled with said shaft, said head including a distal face with a plurality of cutting teeth and at least one viewing window, each said viewing window extending through said head;
placing said orthopaedic reamer against the bone;
rotating said orthopaedic reamer to thereby cut the bone using said plurality of teeth;
stopping rotation of said orthopaedic reamer; and
viewing the bone through at least one said viewing window.

10. The method of claim 9, wherein the bone is associated with one of a shoulder, knee and hip.
